(19) United States
(12) Patent Application Publication
(10) Pub. No.: US 2009/0124891 A1
(43) Pub. Date: May 14, 2009

(54) IMAGE GUIDED SURGERY SYSTEM

(75) Inventors: Guy Shechter, Briarcliff Manor, NY (US); Douglas Stanton, Ossining, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/293,440

(22) PCT Filed: Mar. 19, 2007
(86) PCT No.: PCT/IB2007/050955

§ 371 Date: Sep. 18, 2008

Related U.S. Application Data
(60) Provisional application No. 60/788,441, filed on Mar. 31, 2006.

(51) Int. Cl. A61B 19/00

(52) U.S. Cl. 600/424; 600/427

(57) ABSTRACT

An image guided surgery system is disclosed that includes a position detection system which measures the position of a surgical instrument and displays the surgical instrument in its corresponding position in a CT-image or an MRI-image. The position detection system is provided with an indicator system which shows a region for which the position detection system is sensitive. Preferably, the camera unit of the position detection system incorporates at least two cameras and two semiconductor lasers for emitting separate laser beams that intersect and generate a visible marker within the region, each of the semiconductor lasers being mounted on the camera unit such that each of the laser beams substantially track the optical axis of each camera.

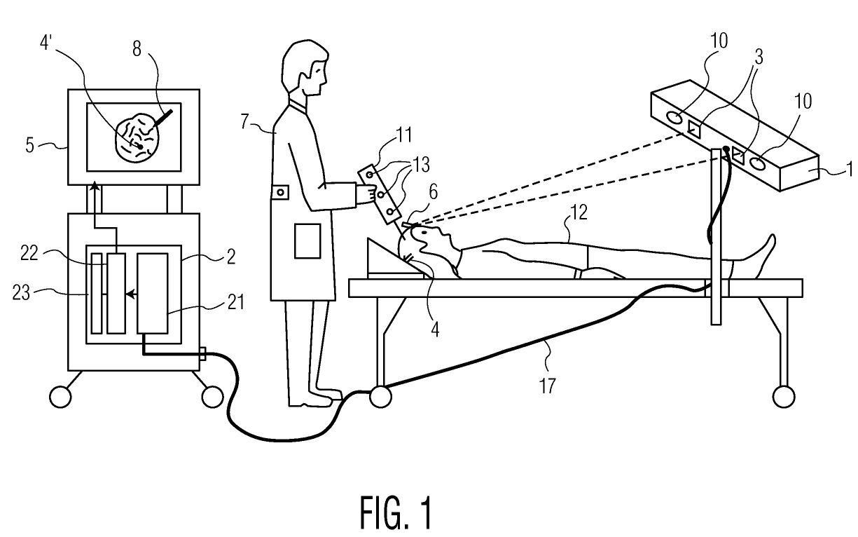

The present disclosure relates to an image guided surgery system that includes an advantageous position detection system.

An image guided surgery system is known from the U.S. Pat. No. 5,389,101.

Image guided surgery systems are generally employed to assist the surgeon to position a surgical instrument during an operation. During complicated surgery it is often very difficult or even impossible for the surgeon to see directly where in the interior of the patient he/she moves the surgical instrument. On a display device the image guided surgery system shows the surgeon the position of a surgical instrument relative to the region where the surgical operation is being performed. Thus, the image guided surgery system enables the surgeon to move the surgical instrument inside the patient and beyond direct sight, without risk of damaging vital parts.

The position detection system of the known image guided surgery system includes two cameras which pick-up images of the surgical instrument from different directions. The image guided surgery system includes a data processor for deriving the position in space of the surgical instrument from image signals from both cameras. During the operation images that had been collected earlier are being shown to the surgeon. For example computed tomography (CT) image or magnetic resonance (MRI) images which were formed before the operation may be displayed on a monitor. The data processor calculates the corresponding position of the surgical instrument in the image. In the displayed image the actual position of the surgical instrument is shown together with an image of a region in which the surgical instrument is used.

Such an image guided surgery system is preferably employed in neuro-surgery to show the surgeon the position of the surgical instrument in the brain of a patient who is being operated on.

A drawback of the known image guided surgery system is that it is difficult to know when the surgical instrument has moved beyond the measuring field. Should the instrument be moved outside the measuring field, then the position detection system will no longer be able to detect the position of the surgical instrument.

In an attempt to overcome this problem, U.S. Pat. No. 5,954,648 discloses an improved image guided surgery system which incorporates an indicator system that can generate a light source, such as from a semiconductor laser.

However, problems still persist. The cameras of the optical tracking or position detection system are usually preconfigured so that their optical axis converge at a nominal distance away from the camera. This convergence point approximately defines the center of the field of view (“sweet spot”) of the optical tracking system. It is difficult to position optimally the camera system in a surgical environment since it is difficult to determine the location of the center of the field of view of the optical tracking system.

In practice, the optical tracking system is first manually positioned in an approximate position, with an initial orientation facing the desired workspace (i.e., operating region). Then the user (e.g., the surgeon) tries to track objects in the desired workspace to test whether the workspace is contained in the field of view of the optical tracking system (i.e., the measuring field). If not, the user makes an adjustment to the position and/or orientation of the tracking system and runs another test. These iterations continue until the orientation and position of the optical tracking system is found to be satisfactory.

Also, U.S. Patent Application Number 2005/0015099 A1 published on Jan. 20, 2005 discloses a surgical position measuring apparatus including at least two laser beams for determining the position of the surgical tool. However, there is no disclosure as to overcoming the problem of rapidly ensuring the camera field of view and the operating region substantially coincide during the operative procedure.

An object of the present disclosure is to provide an image guided surgery system that includes, inter alia, a position detection system that can be accurately directed to the operating region.

This object is achieved by an image guided surgery system according to the present disclosure which is characterized in that the position detection system is provided with an indicator system having a plurality of semiconductor lasers, e.g., two semiconductor lasers, for marking a region for which the position detection system is sensitive.

The operating region is a space in which the surgical instrument is moved during the surgical treatment. The indicator system shows, relative to the operating region, the portion of space for which the position detection system is sensitive, i.e., the measuring field of the position detection system. The measuring field is the part of space from which the camera unit picks-up images. The position detection system is directed by arranging the camera unit and the operating region relative to one another.

Preferably, the camera unit is directed to the operating region, but the patient to be operated on may also be moved so as to move the operating region within the measuring field of the position detection system. The indicator system shows whether or not the measuring field adequately corresponds with the operating region. The camera unit of the position detection system is easily accurately directed in that the region for which the position detection system is sensitive, i.e., such that the measuring field substantially corresponds with the operating region. Hence, complications, which would occur due to the surgical instrument leaving the measuring field are easily avoided. This reduces stress on the surgeon performing an intricate operation. Moreover, the image guided surgery system according to the present disclosure renders unnecessary elaborate test runs for accurately directing the camera unit before the actual surgery can be started. The image guided surgery system according to the present disclosure provides these advantages not only for surgical operations of a patient's brain or spinal cord, but also in surgery related to other anatomical regions and/or organs.

A preferred embodiment of an image guided surgery system according to the present disclosure is characterized, at least in part, in that the indicator system is arranged to mark the center of said region.

In such preferred/exemplary embodiments, the indicator system shows the center, that is a position substantially in the middle, of the measuring field. The position detection system is accurately directed to the operating region when the center shown by the indicator system falls substantially together with the center of the operating region. As an alternative, the indicator system is arranged to show a boundary of the measuring field. In the latter case, the position detection system is accurately directed to the operating region when the boundaries of the measuring field are shown to encompass the operation region.

A further preferred embodiment of an image guided surgery system according to the present disclosure is characterized, at least in part, in that the indicator system is arranged to provide a rendition of said region on a display device.

A rendition of said region on a display device field is, for example, a center showing the circumference of the measuring field, or a sign indicating the center of the measuring field. The rendition of the measuring field is typically displayed on the display device together with the operating region. Hence, it is easy to accurately direct the position detection system such that the measuring field corresponds to the operating region. Namely, while the position detection system is being aligned, the actual measuring field is being displayed together with the operating region. Hence, the display device shows how the measuring field is brought into correspondence with the operating region.

A further preferred embodiment of an image guided surgery system according to the present disclosure is characterized, at least in part, in that the indicator system is arranged to measure an operating region.

In such preferred/exemplary embodiments, the indicator system is arranged to detect a light source that is placed in the operating region in which the surgical instrument is going to be moved. In such embodiment(s), the camera unit of the position detection system is also generally employed to detect the light source as well. Instead of using a separate light source, the patient who is to be operated on may be detected. In that case, preferably an infrared camera, which may also be a camera of the position detection system, is employed. The indicator system is further arranged to display the image of the light source or the patient himself on the display device. When the measuring field does not sufficiently correspond to the operating region, then the indicator system will not be able to detect the light-source or the patient. When there is only little overlap of the measuring field with the operating region, then the light source or the patient will be detected in a peripheral region of the measuring field.

A further preferred embodiment of an image guided surgery system according to the present disclosure is characterized, at least in part, in that the indicator system is arranged to generate a visible marker (i.e., the point of intersection of two laser beams) in a region of interest.

The visible marker shows where the measuring field is. In particular, the visible marker shows the center of the measuring field. Thus, the location of the measuring field is indicated.

A further preferred embodiment of an image guided surgery system according to the present disclosure is characterized, at least in part, in that the indicator system includes two semiconductor lasers for emitting separate laser beams that intersect and generate a visible marker within the measuring region, each of the semiconductor lasers being mounted on the camera unit such that each of the laser beams would substantially track the optical axis of each camera.

The intersecting laser light beams fall on the operating region and generate a light spot, which forms a visible marker. Preferably, the intersection point of the laser light beams is located in the center of the measuring field. The light spot shows the center of the measuring field in the operating region. For example, when the image guided surgery system is employed in brain surgery, the position detection system is accurately directed when the light spot falls at a suitable position of the patient's head. Such suitable positions include, for example, the middle of the patient's head, or a position slightly above that middle. The surgeon or an assistant who chooses the position where the light spot should fall takes into account the region in which the operation is going to be performed. Moreover, it is avoided that the measuring field of the camera unit is obstructed by any equipment that is placed next to the image guided surgery system.

A semiconductor laser emits a narrow beam of light. Moreover, a semiconductor laser is generally relatively inexpensive and has a low power consumption. Preferably, a Class I semiconductor laser is employed which is harmless for the patient and staff and which emits visible light.

These and other aspects of the present disclosure are explained in more detail with reference to the following embodiments and with reference to the drawing.

To assist those of ordinary skill in the art in making and using the disclosed system, reference is made to the accompanying FIGURE.

The drawing comprises one FIGURE which shows a schematic diagram of an image guided surgery system according to the invention.

The FIGURE shows a schematic diagram of an exemplary image guided surgery system according to the present disclosure. The image guided surgery system includes a position detection system which includes a camera unit 1 with at least two cameras 10 and a data processor 2. The cameras pick up images from different directions of a surgical instrument 11. For example, the camera unit 1 incorporates two CCD image sensors mounted on a rigid frame. The frame is moveable so as to direct the CCD sensors to the operating region. The image signals from separate cameras, or subsequent image signals from the cameras but from successive camera positions, are supplied to the data processor 2. To that end, the camera unit 1 is coupled to the data processor 2 by way of a cable 17. The data processor 2 includes a computer 21 which, on the basis of the image signals, computes the position of the surgical instrument relative to the patient 12 who is undergoing a surgical operation. The image processor 22 is incorporated in the data processor 2. The surgical instrument is fitted with light or infrared emitting diodes 13 (LEDs or IREDs) which emit radiation for which the cameras 10 are sensitive. The computer 21 also computes the corresponding position of the surgical instrument 11 in an earlier generated image, such as a CT image or an MRI image. The CT data and/or MRI data are stored in a memory unit 23.

In the image data, fiducial markers are imaged which are placed on particular positions on the patient. For example, lead or MR-susceptible markers are placed at the ears, nose and forehead of the patient. At the start of the operation the fiducial markers are indicated with a surgical instrument filled with LEDs or IREDs and their positions in space are measured by the position detection system. The computer 21 calculates the transformation matrix which connects the positions in space of the fiducial markers to the corresponding positions of the images of the markers in the earlier generated image. This transformation matrix is subsequently used to compute a corresponding position in the image for any arbitrary position in space in the actual operating region.

The data from the memory unit 23 are supplied to the image processor 22. The position-data computed by the computer 21 are also supplied to the image processor 22. The computer 21 may be alternatively programmed to calculate the coordinates of the position of the surgical instrument with respect to as fixed reference system, then the image processor 22 is arranged to convert those coordinates to the corresponding position in the image. The image processor is further arranged to select an appropriate set of image data on the basis of the position of the surgical instrument. Such an appropriate set, e.g., represents CT or MRI image data of a particular slice through the operating region. The image processor 22 generates an image signal which combines the earlier generated image data with the corresponding position of the surgical instrument. In a rendition of the earlier generated image information, also the corresponding position of the surgical instrument is displayed.

Thus, the surgeon 7 who handles the surgical instrument 11 can see the actual position of the surgical instrument 11 in the operating region on the display device 5. On the display device 5, e.g., a CT-image is shown with an image 8 of the surgical instrument in the corresponding positive in the CT-image. Thus, the position of the surgical instrument in the operating region is shown on the display device 5. The display device is, e.g., a monitor that includes a cathode-ray tube, but an LCD display screen may be used as well.

The camera unit 1 includes an indicator system which, for example, includes two semiconductor lasers 3. The semiconductor lasers 3 are each mounted on the camera unit adjacent the cameras 10, and positioned and oriented so that the emitted laser beams will approximate and track the optical axis of each camera and will intersect, thereby generating at the point of intersection a visible marker within the measuring field. Each semiconductor laser emits a narrow light beam through the measuring field of the camera unit. Thus, the system of the present disclosure simplifies the setup of the position detection system in a medical/surgical environment. The user/surgeon can quickly observe the intersection spot of the laser beams, and position the camera unit of the position detection system (i.e., optical tracking system) so that the intersection spot 6 is located on the patient's body in the operating region, ensuring the measuring field of the cameras substantially overlaps the operating region. To accurately direct the camera unit so that the measuring field of the camera unit covers the operating region, the light spot 6 is positioned at the center of the operating region.

In this way, it is achieved that the measuring field extends in about the same amount in all directions from the center of the operating region. Hence, the risk that the surgical instrument is moved beyond the measuring field of the camera unit is significantly reduced and/or completely eliminated. Moreover, it is avoided that the measuring field of the camera unit is obstructed by any equipment that is placed next to the image guided surgery system. Namely, should some equipment be placed between the camera unit and the operating region, then the intersecting laser beams generate the light spot 6 on the equipment that is in the way rather than on the patient. Hence, the person directing the camera unit is immediately made aware that equipment is blocking the measuring field of the camera unit and that equipment should be re-arranged before starting surgery.

Additionally, the indicator system may include a radiation source 4 that is positioned at the operating region. With the cameras 10 the radiation source 4 is observed. The image signals of the cameras are processed by the computer 21 and by the image processor 22. An image 4 of the radiation source is displayed on the display device 5. Preferably, the image processor 22 and the monitor 5 are arranged such that the center of the measuring field of the camera unit 1 is displayed in the center of the display screen of the monitor 5. Then, the camera unit 1 is accurately directed when the radiation source 4 is imaged in the middle of the display screen. Preferably, an infrared emitting diode (IRED) is employed as a radiation source, such IRED emitting infrared radiation for which the cameras 10 are substantially sensitive. Instead of a separate IRED, also the patient itself may be employed. In that case, the cameras 10 pick-up infrared images of the patient which are displayed on the monitor.

Position detection systems or optical tracking systems are used to locate objects in space. Two or more cameras observe the target object and triangulate its position in 3D space. Commercial products include the Polaris and Certus System made by Northern Digital Inc., Waterloo, Ontario. These systems have a limited field of view. In practice, one has to set-up the tracking system so that its field of view covers the intended work environment. For example, suppose one wanted to track the position of a laparoscope and/or endoscope being inserted into a patient's abdomen in a surgical suite. The optical tracking system would have to be positioned in a location such that its field of view covered the area around the patient's abdomen. Thus, this positioning problem is overcome in accordance with the disclosed invention herein.

Consider the use of the image guided system of the invention herein for a tumor biopsy in a human liver. The position detection system would be used to track the patient and position of the biopsy needle. The user would turn on the lasers and look for the point at which the laser beams intersect. The user would then orient and reposition the position detection system so that the point of intersection would coincide with the position of the patient's liver. Thus, the patient's liver could be quickly positioned in the center of the field of view of position detection system.

Other examples of use of the image guided system of the present disclosure include positioning and/or orienting the use of medical needles or catheters; and use with portable and rotational x-ray imaging systems and handheld ultrasound transducers.

Although the position detection system has been described heretofore utilizing an optical system incorporating a camera unit as one embodiment of the receptor means for receiving a signal from the object whose position is being tracked, it is contemplated within the framework of the present disclosure that other receptor means can also be used that are well known in the art. For example, besides cameras for receiving visual or optical signals, such receptor means for imaging can receive ultrasonic signals (see, e.g., U.S. Pat. Nos. 5,563,346 and 5,511,423); magnetic or electromagnetic signals (see, e.g., U.S. Pat. Nos. 7,003,342; 6,990,417; and 6,856,823); and radio frequency (RF) signals (see, e.g., U.S. Pat. No. 6,762,600).

While the present invention has been described with respect to specific embodiments thereof, it will be recognized by those of ordinary skill in the art that many modifications, enhancements, and/or changes can be achieved without departing from the spirit and scope of the invention. Therefore, it is manifestly intended that the invention be limited only by the scope of the claims and equivalents thereof.

1. An image guided system comprising:
a position detection system for detecting a position of a surgical instrument in an operating region of a patient to be operated on, the position detection system comprising a receptor means for picking up signals, a memory unit for storing an image of a patient, and data processor means for processing signals from the receptor means to detect the position of the surgical instrument and for superimposing a detected position of the surgical instrument on the stored image of the patient;
an indicator system for marking a measuring region of the operating region, the position detection system being sensitive in the measuring region; wherein the indicator system comprises two semiconductor lasers for emitting separate laser beams that intersect and generate a visible marker within the measuring region, each of the semiconductor lasers being mounted in close proximity to the receptor means such that each of the laser beams substantially track the signal receiving axis of the receptor means; and
a display for displaying the stored image of the patient with the superimposed detected position of the surgical instrument.

2. The image guided system of claim 1, further comprising:
a position detection system for detecting a position of a surgical instrument in an operating region of a patient to be operated on, the position detection system comprising a camera unit having at least two cameras for picking up image signals, a memory unit for storing an image of a patient, and data processor means for processing image signals from the camera unit to detect the position of the surgical instrument and for superimposing a detected position of the surgical instrument on the stored image of the patient;
an indicator system for marking a measuring region of the operating region, the position detection system being sensitive in the measuring region; wherein the indicator system comprises two semiconductor lasers for emitting separate laser beams that intersect and generate a visible marker within the measuring region, each of the semiconductor lasers being mounted on the camera unit such that each of the laser beams substantially track the optical axis of each camera;
and a display for displaying the stored image of the patient with the superimposed detected position of the surgical instrument.

3. The image guided system of claim 1, wherein the visible marker is generated in the center of the measuring region.

4. The image guided system of claim 1, wherein the data processor means are also for superimposing a sign indicating the center of the measuring region on the stored image of the patient.

5. The image guided system of claim 1, wherein the data processor means are also for superimposing a contour indicating the circumference of the measuring region on the stored image of the patient.

6. The image guided system of claim 1, wherein the indicator system further comprises means to detect a current image of the patient, and wherein the data processor means are also for superimposing the current image of the patient on the stored image of the patient.
